(19) United States
(12) Patent Application Publication
(10) Pub. No.: US 2009/0131899 A1
(43) Pub. Date: May 21, 2009

(54) HYGIENE PAD

(75) Inventor: Linda Ross, Detroit, MI (US)

Correspondence Address: BROOKS KUSHMAN P.C., 1000 TOWN CENTER, TWENTY-SECOND FLOOR, SOUTHFIELD, MI, 48075, US

(73) Assignee: DERRIAY, INC., Detroit, MI (US)

(21) Appl. No.: 12/273,181

(22) Filed: Nov. 18, 2008

Related U.S. Application Data
(60) Provisional application No. 61/003,934, filed on Nov. 20, 2007.

(51) Int. Cl. A61F 13/15

(52) U.S. Cl. 604/385.03; 604/385.01

(57) ABSTRACT

A hygiene pad and method of collecting bodily fluids from a user are provided, where the hygiene pad includes a body having a top surface, an opposed bottom surface, and an edge extending around a periphery of the body between the top and bottom surfaces. The edge includes a first side having first and second ends and a second side connected to the first and second ends, where the first side may be generally straight or generally concave, and the second side may be generally curved or convex. The hygiene pad is inserted into a perineum cleft of the user posterior to the genital area and anterior to the anus to collect bodily fluids of the user.

### CROSS-REFERENCE TO RELATED APPLICATIONS

This application claims the benefit of U.S. provisional application Ser. No. 61/003,934 filed Nov. 20, 2007, which is incorporated by reference herein in its entirety.

### BACKGROUND OF THE INVENTION

1. Field of the Invention

This invention relates to hygiene pads and methods of their use.

2. Background Art

Devices designed to absorb bodily fluids and protect the undergarments and clothing from staining include hygiene products such as tampons, sanitary napkins and absorbent pads for use during menstruation, as well as absorbent pads for urinary and fecal incontinence. Although absorbent pads are typically designed to fit the average person, these pads have not always provided the protection required. The fit of the absorbent pad is difficult to maintain when moving about during the day. Standing, bending and sitting may result in rounding of the pad which, in turn, results in less contact along the contour of the body. Bodily fluids which do not contact the pad follow the contour of the body and tend to pool in the perineum cleft between the genital area and the anus. Smaller hygiene products such as tampons and rectal pads also leak when there is an increased flow of bodily fluids beyond the absorbency level of the hygiene product or due to delayed time between changes.

### BRIEF DESCRIPTION OF THE DRAWINGS

FIG. 1 is a perspective view of a hygiene pad according to one aspect of the present invention;

FIG. 2 is a side elevational view of the hygiene pad of FIG. 1;

FIG. 3 is a top plan view of the hygiene pad of FIG. 1;

FIG. 4 is a bottom plan view of the hygiene pad of FIG. 1;

FIG. 5 is a perspective view of a hygiene pad according to another aspect of the present invention;

FIG. 6 is a side elevational view of the hygiene pad of FIG. 5;

FIG. 7 is a top plan view of the hygiene pad of FIG. 5;

FIG. 8 is a perspective view illustrating the hygiene pad of FIG. 5 folded along its transverse axis;

FIG. 9 is an illustration of the position of a hygiene pad in relation to the human body according to one aspect of the present invention;

FIG. 10 is an illustration of the position of a hygiene pad in relation to the human body according to another aspect of the present invention; and

FIG. 11 is a perspective view of a hygiene pad in accordance with another aspect of the present invention.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for the claims and/or as a representative basis for teaching one skilled in the art to variously employ the present invention.

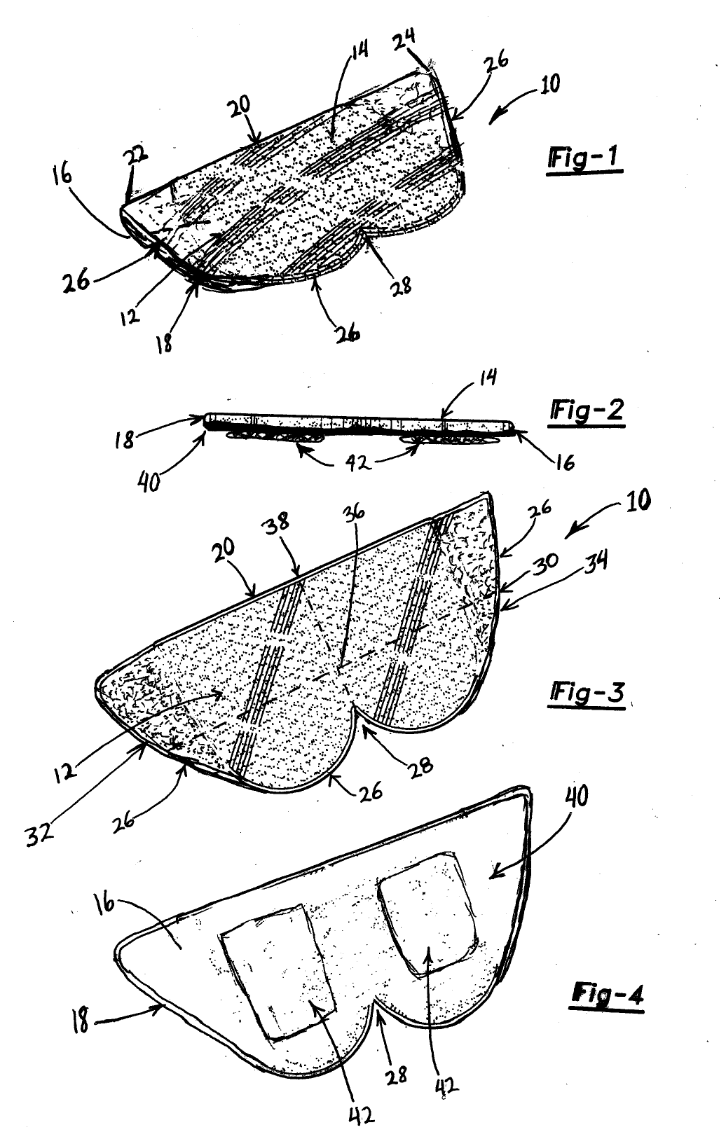

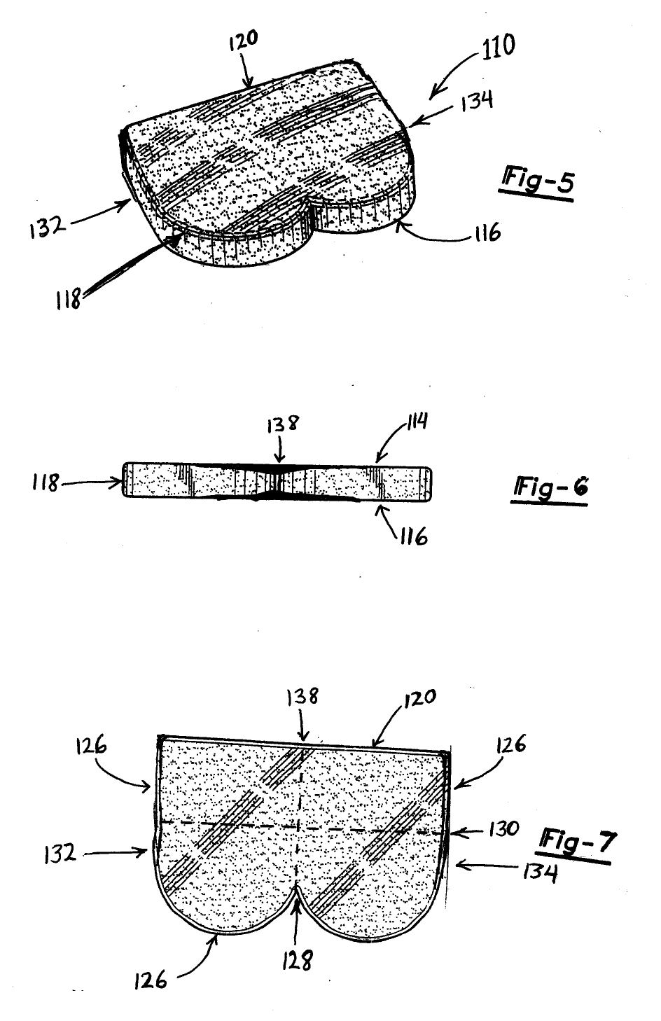

With reference to FIG. 1, a hygiene pad according to one aspect of the present invention is illustrated and designated generally by reference numeral 10. The hygiene pad 10 according to the present invention may be utilized in a stand-alone manner or may be utilized as an auxiliary device in conjunction with a primary hygiene product, such as another pad or tampon, for collecting bodily fluids which may overflow from the primary product. The hygiene pad 10 includes a body 12 having a top surface 14 an opposed a bottom surface 16. The hygiene pad 10 further includes an edge 18 extending around a periphery of the body 12 between the top and bottom surfaces 14, 16. The edge 18 includes a first side 20 having a first end 22 and a second end 24, and a second side 26 connected to the first and second ends 22, 24. According to one aspect of the present invention, the first side 20 may be generally straight, and the second side 26 may be generally curved or convex. The second side 26 may include an indentation 28 therein, such that the body 12 may generally have a B-shaped appearance when viewed in plan (see FIGS. 3 and 4).

According to another aspect of the present invention, as depicted in FIG. 11, the hygiene pad 10 may be constructed such that the first side 20 is generally concave and the second side 26 is generally convex. It is understood that the terms “concave” and “convex” as used herein are not restricted to describing sides with a constant radius of curvature, but rather are used to denote the general appearance of the sides. As illustrated in both FIGS. 1 and 11, the hygiene pad 10 may be constructed with one or more indentations 28, however, the indentation 28 is not required.

As depicted in FIG. 3, the body 12 includes a longitudinal axis 30 extending generally parallel to the first side 20 and intersecting the second side 26 to define a front end 32 and a back end 34 of the body 12. In this embodiment, a length of the first side 20 is greater than a distance between the front and back ends 32, 34. According to one aspect of the present invention, the indentation 28 may be located at a midpoint of the second side 20, which is aligned with a midpoint 36 of the longitudinal axis 30. The body 12 also includes a transverse axis 38 extending from the second side 26 to the first side 20 through the midpoint 36, wherein the body 12 may be generally symmetric about the transverse axis 38. In one embodiment, the first side 20 may have a length of approximately 10 cm to 15 cm, and a distance from the indentation 28 to the first side 20 along the transverse axis 38 may be approximately 4 cm to 8 cm. Additionally, the hygiene pad 10 may have a thickness of approximately 0.25 cm to 2 cm. Of course, the hygiene pad 10 according to the present invention is not limited to these dimensions, and other dimensions are fully contemplated.

According to one aspect of the present invention, the body 12 may be at least partially constructed from an absorbent material which may include, but is not limited to, biodegradable cotton, synthetic polymer, pulp paper and mixtures thereof. Of course, other materials may alternatively be utilized to construct the body 12 according to the present invention. The material(s) selected for construction of the hygiene pad 10 may also be flushable. Still further, the material(s) of construction may be capable of containing medication or other solid or liquid formulations which may be desired for administration, such as by contact with a user's skin. The body 12 may also be at least partially enclosed by an absorbent woven covering (see FIG. 3).

As depicted in FIGS. 2 and 4, the bottom surface 16 may be constructed to include a non-absorbent material 40, which may act as a barrier to prevent collected fluids from penetrating through the bottom surface 16. The non-absorbent material 40 may contain, but is not limited to, thermoplastics, polyvinylidene chloride, low density polyethylene (LDPE), high density polyethylene (HDPE), and mixtures thereof. In another embodiment, the bottom surface 16 may include an adhesive material 42 which may provide a temporary attachment between the hygiene pad 10 and a primary hygiene product or the user's clothing. The adhesive material 42 may be at least partially composed of natural polymers, synthetic chemicals or mixtures thereof, but is not limited to these materials. The adhesive material 42 may be protected by a removable cover (not shown) until the hygiene pad 10 is utilized.

Referring now to FIGS. 5-7, a hygiene pad in accordance with another aspect of the present invention is designated generally by 110, wherein components of the hygiene pad 110 corresponding to the hygiene pad 10 of FIG. 1 have like reference numerals with the addition of a “1” prefix. Hygiene pad 110 includes a thicker edge 118 as compared with edge 18, which may be beneficial if greater absorbency is required. In this embodiment, a length of the first side 120 is approximately equal to a distance between the front and back ends 132, 134. It is understood that the length of the first side 20, 120 may be greater than, approximately equal to, or less than the distance between the front end 32, 132 and back end 34, 134 in accordance with the present invention. In addition, the bottom surface 116 may taper near the transverse axis 138, as shown in FIG. 6, which may facilitate folding of the hygiene pad 10, 110 as described below. It is understood that the features shown and described for the embodiments of FIGS. 1, 5, and 11 are interchangeable.

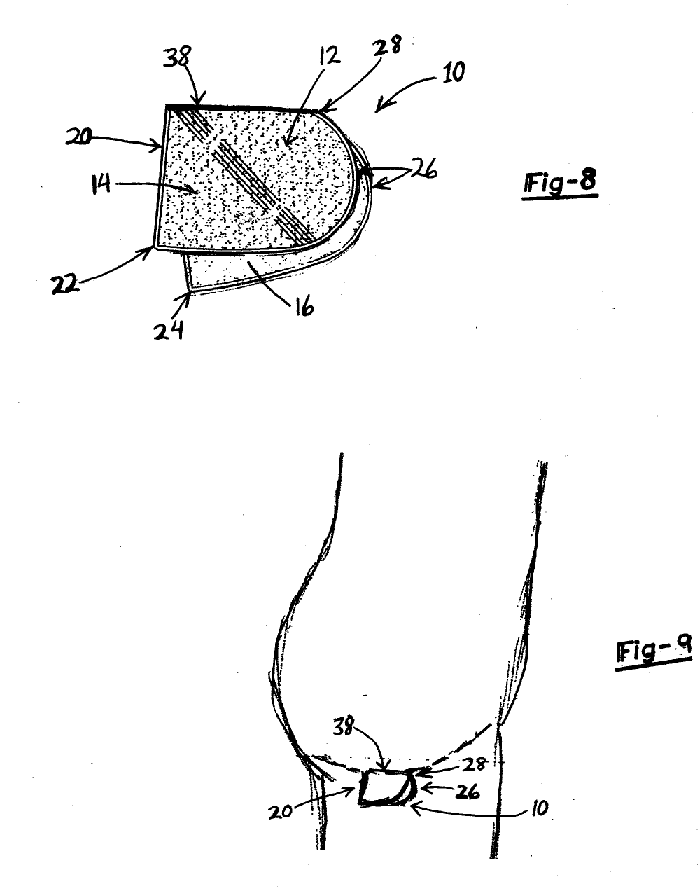

In further accordance with the present invention, the hygiene pad 10, 110 may be used for collecting bodily fluids from a user. Bodily fluids may include, but are not limited to, menstrual flow, vaginal discharge, urine, fecal matter or any combination thereof. Fluids may also include medications or solid or liquid formulations. As depicted in FIG. 8, the hygiene pad 10, 110 may be folded to bring the first end 22, 122 and second end 24, 124 toward each other, such that the folded hygiene pad 10, 110 resembles an inverted V. In this configuration, the top surface 14, 114 may be on the exterior, although this is not required. The hygiene pad 10, 110 may be folded along the transverse axis 38, 138 as shown, although other folding locations are also contemplated. The folded hygiene pad 10, 110 may then be inserted into a perineum cleft of the user posterior to the genital area and anterior to the anus to collect bodily fluids of the user as illustrated in FIG. 9. According to one aspect of the present invention, the body 12, 112 may be grasped along either the first side 20, 120 or the second side 26, 126 and inserted such that the transverse axis 38, 138 contacts the perineum cleft.

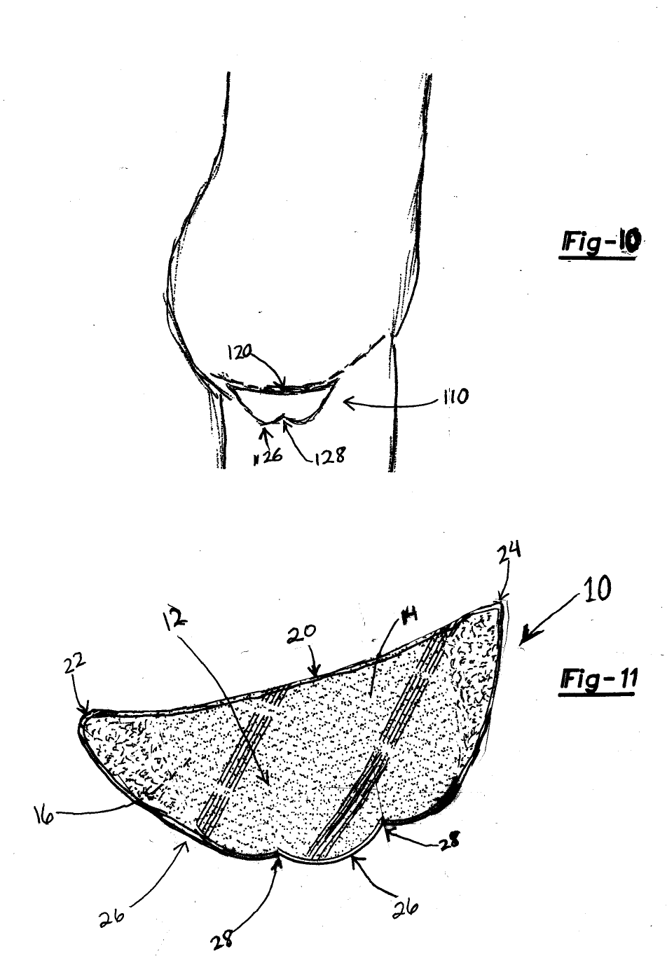

Of course, the hygiene pad 10, 110 is not limited to this method of insertion. For example, as depicted in FIG. 10, the hygiene pad 10 may alternatively be inserted into the perineum cleft without folding, such that a user may grasp the second side 26, 126 and orient the first side 20, 120 to contact the perineum cleft. In this embodiment, a relatively longer first side 20, 120 may be beneficial to draw fluids into the hygiene pad 10, 110 from a larger area along the perineum cleft. The generally concave first side 20 illustrated in the embodiment of FIG. 11 may be beneficial for following the contour of the user's body when inserted. The hygiene pad 10, 110 may be held in place by the anatomy of the body, or may be adhered to undergarments or a primary hygiene product with adhesive material 42, as described above and depicted in FIG. 4. Again, although hygiene pad 10 is depicted in FIG. 9 and pad 110 is depicted in FIG. 10, it is understood that either pad 10, 110 may be used for either method of insertion.

The hygiene pad 10, 110 may be positioned to capture bodily fluids which follow the contour of the human body into the perineum cleft from the genital or anal area. In the embodiment depicted in FIG. 8, the curved second side 26, 126 and associated indentation 28, 128 may act as a channel for collection of the bodily fluids into the hygiene pad 10, 110. Thus, the hygiene pad 10, 110 may be oriented such that the curved second side 26, 126 faces either anteriorly or posteriorly within the perineum cleft depending on the origin of the bodily fluid, either the genitals or the anus. The hygiene pad 10, 110 according to the present invention may be utilized in a stand-alone manner or may be utilized as an auxiliary device in conjunction with a primary hygiene product, such as another pad or tampon.

While embodiments of the invention have been illustrated and described, it is not intended that these embodiments illustrate and describe all possible shapes and forms of the invention. Rather, the words used in the specification are words of description rather than limitation, and it is understood that various changes may be made without departing from the spirit and scope of the invention.

What is claimed is:

1. A hygiene pad, comprising:
a body having a top surface, an opposed bottom surface, and an edge extending around a periphery of the body between the top and bottom surfaces, the edge including a generally straight first side having first and second ends and a curved second side connected to the first and second ends.

2. The hygiene pad according to claim 1, wherein the body includes a longitudinal axis extending generally parallel to the first side and intersecting the second side to define a front end and a back end of the body, wherein a length of the first side is greater than a distance between the front and back ends.

3. The hygiene pad according to claim 1, wherein the body includes a longitudinal axis extending generally parallel to the first side and intersecting the second side to define a front end and a back end of the body, wherein a length of the first side is approximately equal to a distance between the front and back ends.

4. The hygiene pad according to claim 1, wherein the body is at least partially constructed from an absorbent material.

5. The hygiene pad according to claim 4, wherein a thickness of the absorbent material is tapered along the bottom surface.

6. The hygiene pad according to claim 1, wherein the bottom surface includes a non-absorbent material.

7. The hygiene pad according to claim 1, wherein the bottom surface includes an adhesive material.

8. The hygiene pad according to claim 1, wherein the second side includes at least one indentation.

9. The hygiene pad according to claim 1, wherein the body includes a transverse axis extending from a midpoint of the second side to the first side, and wherein the body is symmetric about the transverse axis.

10. The hygiene pad according to claim 9, wherein a distance from the midpoint of the second side to the first side along the transverse axis is approximately 4 cm to 8 cm.

11. The hygiene pad according to claim 1, wherein a length of the first side is about 10 to 15 cm.

12. The hygiene pad according to claim 1, wherein the body is approximately 0.25 cm to 2 cm thick.

13. A hygiene pad, comprising:
a body having a top surface, an opposed bottom surface, and an edge extending around a periphery of the body between the top and bottom surfaces, the edge including a generally concave first side having first and second ends and a generally convex second side connected to the first and second ends.

14. A method of collecting bodily fluids from a user, comprising:
providing a hygiene pad including a body having a top surface, an opposed bottom surface, and an edge extending around a periphery of the body between the top and bottom surfaces, the edge including a generally straight first side having first and second ends and a curved second side connected to the first and second ends; and
inserting the hygiene pad into a perineum cleft of the user posterior to the genital area and anterior to the anus to collect bodily fluids of the user.

15. The method according to claim 14, wherein the first side contacts the perineum cleft upon insertion of the hygiene pad.

16. The method according to claim 14, further comprising folding the hygiene pad to bring the first and second ends toward each other prior to insertion.

17. The method according to claim 14, wherein the second side includes an indentation at a midpoint thereof, and folding the hygiene pad includes folding the body along a transverse axis extending from the indentation to the first side.

18. The method according to claim 17, wherein the transverse axis contacts the perineum cleft upon insertion of the hygiene pad.

19. The method according to claim 18, further comprising orienting the second side toward a source of the bodily fluids.

20. A method of collecting bodily fluids from a user, comprising:
providing a hygiene pad including a body having a top surface, an opposed bottom surface, and an edge extending around a periphery of the body between the top and bottom surfaces, the edge including a generally concave first side having first and second ends and a generally convex second side connected to the first and second ends; and
inserting the hygiene pad into a perineum cleft of the user posterior to the genital area and anterior to the anus to collect bodily fluids of the user.
